(19) United States
(12) Patent Application Publication
(10) Pub. No.: US 2026/0102539 A1
(43) Pub. Date: Apr. 16, 2026

(54) ANTIBIOFILM AND ANTIMICROBIAL SURFACES

(71) Applicant: BRIGHAM YOUNG UNIVERSITY, Provo, UT (US)

(72) Inventors: Bradford K. Berges, Provo, UT (US); Lucy C. Bowden, Provo, UT (US)

(21) Appl. No.: 19/245,063

(22) Filed: Jun. 20, 2025

Related U.S. Application Data
(60) Provisional application No. 63/667,290, filed on Jul. 3, 2024.

(51) Int. Cl. A61L 27/30; A01N 59/00; A01N 59/20; A01P 1/00; A61L 27/54

(52) U.S. Cl. CPC A61L 27/306; A01N 59/00; A01N 59/20; A01P 1/00; A61L 27/303; A61L 27/54; A61L 2420/02; A61L 2420/08

(57) ABSTRACT

Systems and methods for providing an antibiofilm, antimicrobial coating are disclosed. In some implementations, the coating includes a nanotexture having an anti-bacterial topology, and in some implementations, the coating includes an antimicrobial layer having antibacterial characteristics. In some cases, the antimicrobial layer is formed on the nanotexture, and in some implementations, the two work synergistically to produce a compounding effect to drastically reduce the viability of one or more microorganisms. According to some implementations, the nanotexture includes carbon-infiltrated carbon nanotubes, and according to some implementations, the antimicrobial layer includes copper. Additional implementations are described.

### RELATED APPLICATION AND PRIORITY CLAIM

This application claims priority to U.S. Provisional Application Ser. No. 63/667,290, filed Jul. 3, 2024, and entitled “Development of antimicrobial surfaces that prevent biofilm formation”; the entire disclosure of which is hereby incorporated herein by reference.

### FIELD

The present disclosure relates to antibiofilm and antimicrobial surfaces and materials. More specifically, it pertains to surfaces or coatings on substrates that can reduce or eliminate bacterial colonization by inhibiting biofilm formation.

### BACKGROUND

Bacterial infections are often a major concern in clinical settings, particularly in connection with surgical procedures and the use of implanted medical devices. Once introduced into the body, bacteria can adhere to tissue or foreign materials and begin forming biofilms or structured communities of microbial cells that can be encased in a protective extracellular matrix.

Biofilms present unique and serious challenges to patient health. Unlike planktonic bacteria, biofilm-associated bacteria typically exhibit heightened resistance to antibiotics and are largely impervious to host immune responses. As a result, infections involving biofilms are notoriously difficult to treat and often persist despite aggressive therapeutic intervention.

When biofilms develop on medical implants or otherwise in connection with a living subject, they can lead to chronic infections, device failure, systemic complications, or other serious complications. These infections may require surgical revision, device removal, or long-term antibiotic therapy, all of which can carry substantial clinical and economic burdens. Moreover, in some cases, antibiotic therapy or other existing methods of treating biofilms can be ineffective, thus further increasing the danger and costs associated with such biofilms.

The prevalence of biofilm-related complications underscores the need for improved approaches to preventing bacterial colonization, particularly in the context of surgically implanted materials. Thus, while techniques currently exist that are used to provide implants and treat bacterial infections, challenges still exist, including those listed above. Accordingly, it would be an improvement in the art to augment or even replace current techniques with other techniques.

### SUMMARY

Systems and methods are provided for forming antimicrobial and antibiofilm surfaces. In some implementations, a substrate—such as a medical implant, surgical instrument, or other material configured for use in a biological, clinical, industrial, or other setting—is coated with one or more coatings, comprises surface modifications, or is otherwise configured to reduce bacterial colonization. In some cases, the coating includes a nanotexture, such as a carbon-infiltrated carbon nanotube (CICNT) structure or other nanostructure. In some cases, the coating includes one or more antimicrobial layers, such as a conformal copper coating or another material having antimicrobial activity. According to some implementations, a nanotexture and an antimicrobial layer are combined for a synergistic effect.

In some implementations, the coating is configured to reduce bacterial adhesion and biofilm formation by creating topological conditions that inhibit microbial growth, including for pathogens such as S. aureus, P. aeruginosa, and other bacteria and microorganisms. In some implementations, the nanotexture and antimicrobial layer are configured to act synergistically, resulting in significant or complete reductions in viable microbes compared to uncoated or singly coated surfaces. In some cases, the antimicrobial layer retains the underlying geometry of the nanotexture, preserves coating conformity, and provides long-term activity under biological or environmental stress.

Methods for forming the coatings include thin-film deposition techniques, carbon nanotube growth processes, and combinations thereof. The systems and methods described herein may be adapted or modified in any suitable manner and may be used in connection with a wide variety of materials, substrates, applications, or use cases. Additional (or more detailed) systems and methods are also provided, as described below.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1A shows a top-down scanning electron microscopy (SEM) image of a bare titanium substrate, in accordance with some embodiments of the disclosed systems and methods;

FIG. 1B shows a top-down SEM image of a copper-coated titanium substrate, in accordance with some embodiments;

FIG. 1C shows a top-down SEM image of a carbon-infiltrated carbon nanotube (CICNT) coating, in accordance with some embodiments;

FIG. 1D shows a top-down SEM image of a copper-coated carbon-infiltrated carbon nanotube (Cu-CICNT) coating, in accordance with some embodiments;

FIG. 1E shows a side-view (80° tilt) SEM image of a bare titanium substrate, in accordance with some embodiments;

FIG. 1F shows a side-view (80° tilt) SEM image of a copper-coated titanium substrate, in accordance with some embodiments;

FIG. 1G shows a side-view (80° tilt) SEM image of a CICNT coating, in accordance with some embodiments;

FIG. 1H shows a side-view (80° tilt) SEM image of a Cu-CICNT coating, in accordance with some embodiments;

FIG. 2 shows a graph with various measurements of colony-forming units (CFU) per sample over time+/−standard error for adherent S. aureus grown on bare titanium, CICNT, copper-coated titanium (Cu—Ti), and Cu-CICNT at 6 hours, 12 hours, and 36 hours, with a horizontal line indicating the limit of detection (*p<0.01, **p<0.001, ***p<0.0001), in accordance with some embodiments;

FIGS. 3A-3H show graphic representations of the results of flow cytometry (following RedoxSensor™ Green and PI straining protocol) after 6 hours of S. aureus biofilms growth on titanium (Ti) or Cu-CICNT surfaces, with the individual figures showing as follows: FIG. 3A shows an unstained control, in accordance with some embodiments; FIG. 3B shows a dead-cell positive control (carbonyl cyanide m-chlorophenyl hydrazone (CCCP) treated), in accordance with some embodiments; FIG. 3C shows live cell positive control in accordance with some embodiments; FIG. 3D shows cell grown on Ti, in accordance with some embodiments; FIG. 3E shows cells grown on Cu-CICNT in accordance with some embodiments; FIG. 3F shows a bar graph representing total bacteria acquired on the flow cytometer from the same volume of sample after normalization with beads, in accordance with some embodiments; FIG. 3G shows a bar graph representing the proportion of dead and live bacterial cells, normalized with bead count, grown on either Ti or Cu-CICNT, in accordance with some embodiments; FIG. 3H shows a bar graph representing a live bacteria count normalized with beads, while in each case, data are presented as mean values and error bars represent standard error from three technical replicates per condition across two independent biological experiments, in accordance with some embodiments (noting that in any of the foregoing figures, bars labeled with different letters indicate statistically significant differences (p<0.05), while shared letters indicate no significant difference, and the asterisk indicates statistically significant differences (p<0.001));

FIGS. 4A-4C show additional graphic representations of flow cytometry results, in this case using forward scatter (FSC) and side scatter (SSC) analysis to identify bacterial populations and debris, with the individual figures as follows: FIG. 4A shows FSC and SSC of bacterial cells grown on Cu-CICNT, in accordance with some embodiments; FIG. 4A(i) shows live and dead gating strategy of bacterial cells grown on Cu-CICNT, in accordance with some embodiments; FIG. 4A(ii) shows live and dead gating strategy of debris grown on Cu-CICNT, in accordance with some embodiments; FIG. 4B shows FSC and SSC of bacterial cells grown on Ti, in accordance with some embodiments; FIG. 4B(i) shows live and dead gating of bacterial cells grown on Ti, in accordance with some embodiments; FIG. 4B(ii) shows live and dead cell gating of debris grown on Ti, in accordance with some embodiments; FIG. 4C shows a bar graph representing proportion of debris from bacterial cells grown on either Cu-CICNT or Ti (with data presented as mean values, and error bars representing standard error from three technical replicates per condition across two independent biological experiments, and with asterisk indicating statistically significant differences (p<0.001)), in accordance with some embodiments;

FIG. 5 shows a bar graph of CFU/sample+/−standard error for adherent S. aureus after 12 hours of growth on bare titanium, CICNT, Cu—Ti, or Cu-CICNT (with a horizontal line indicating the limit of detection, *p<0.01, ***p<0.0001), in accordance with some embodiments;

FIG. 6 shows a bar graph of CFU/sample+/−standard error for adherent P. aeruginosa after 12 hours of growth on bare titanium, CICNT, Cu—Ti, or Cu-CICNT (with a horizontal line indicating the limit of detection), in accordance with some embodiments;

FIG. 7 shows a bar graph of CFU/sample+/−standard error for adherent S. aureus grown on bare titanium, CICNT, or either surface with the addition of 6.2 parts per million (ppm) copper ions (from dissolved CuCl₂) added to the culture media (*p<0.05, differences between other groups are not significant), in accordance with some embodiments;

FIG. 8 shows a bar graph of CFU/sample+/−standard error for adherent S. aureus grown on bare titanium, with conditioned media (CM) first exposed to a Cu-CICNT surface, while normal media (NM) from the same batch was used as a control (*p<0.05), in accordance with some embodiments; and

FIGS. 9A-9D show (in accordance with some embodiments) substrates, and in the cases of FIGS. 9B-9D, the substrate having various coatings, wherein these figures also show graphic representations of biofilm formation on the various surfaces (with the exception of FIG. 9D, which shows no biofilm formation).

### DETAILED DESCRIPTION

A description of embodiments will now be given with reference to the figures. It is expected that the present systems and methods may take many other forms and shapes. Hence the following disclosure is intended to be illustrative and not limiting, and the scope of the disclosure should be determined by reference to the appended claims.

Orthopedic implants and prosthetic devices often greatly improve patient quality of life and movement capacity, and their use is continually increasing. However, as mentioned above, bacterial infections are a major concern in many clinical settings—particularly in connection with surgical procedures and the use of implanted medical devices. Once introduced into the body, bacteria can adhere to tissue or foreign materials and form biofilms. For example, implant-associated infections caused by Staphylococcus aureus (S. aureus) and other bacteria are a growing problem for patients and healthcare systems alike as they can increase surgical risks, operating costs, and expenses relating to time, labor, monitoring, medications, and other aspects of medical procedures. In many cases, S. aureus infections are particularly difficult to treat because they form biofilms that are resistant to both antibiotics and to host organism immune systems. Indeed, recalcitrant implant-associated infections are often treated with a two-step revision surgery (which can be painful, time-consuming, expensive, and likely to increase a patient's odds for complications or a shortened mortality). Embodiments of the instant systems and methods can be used to coat one or more substrates 10 to grant such substrates with antibiofilm, antimicrobial, or other desired properties, thereby helping to defend against S. aureus and other microorganisms.

According to some embodiments, one or more substrates 10 is provided. The substrate 10 can include any suitable substrate that could benefit from increased antibiofilm or antimicrobial properties, such as one or more medical devices, medical implants, medical tools, invasive medical devices, furnishings, fixtures, objects, or other materials configured to be used in, on, or around a medical patient, as well as any other substrate that could benefit from antibiofilm or antimicrobial properties. As non-limiting examples, suitable substrates include one or more: orthopedic implants (e.g., hip implants, knee implants, shoulder implants, spinal implants, bone screws, fixation plates, screws, pins, or other orthopedic implants); dental implants (e.g., posts, abutments, crowns, or other dental implants); vascular devices (e.g., catheters, stents, grafts, vascular access ports, pacemakers, or other vascular devices); neurological implants (e.g., cranial plates, electrodes, brain stimulation leads, or other neurological implants); indwelling catheters and tubes (e.g., endotracheal tubes, nasogastric tubes, or other catheters or tubes); prosthetic devices (e.g., limb prostheses, osseointegrated devices, artificial bones, or other prosthetic devices); surgical tools or instruments (e.g., forceps, clamps, endoscopes, laparoscopic instruments, insertion instruments, deployment instruments, scalpels, sutures, or other surgical instruments); external fixation systems and pins; drug delivery devices (e.g., transdermal patches, implantable pumps, microneedle arrays, or other drug delivery devices); wound care materials (e.g., bandages, dressings, meshes, scaffolds for tissue engineering, or other wound care materials); extracorporeal equipment components (e.g., dialysis tubing, filters, oxygenator surfaces, or other extracorporeal components); biosensors or diagnostic chips that contact biological fluids (e.g., glucose monitors, lactate sensors, blood gas analyzers, electrolyte sensors, troponin sensors, coagulation biosensors, etc.); food-contact surfaces in industrial processing settings (e.g., cutting blades, conveyor belts, mixers, filling nozzles, pasteurization lines, mixing bowls, stirring implements, and other food-processing equipment); packaging materials for medical or food applications (e.g., external packaging, food trays, blister packs, or other packaging or similar materials); surfaces in high-touch public, institutional, or other environments (e.g., hospital bed rails, door handles, sink handles, bathroom surfaces, tray tables, elevator buttons, armrests, hand rails, and other high-touch surfaces); consumer goods and surfaces (e.g., water bottles, cutting boards, kitchen countertops, baby bottles, pacifiers, remote controls, or other consumer materials); water systems (water pipes, drains, filtration membranes, porous substrates, water purification systems, sinks, or other water systems); air filtration systems; scientific equipment (e.g., beakers, vials, scientific equipment surfaces, tools, containers, or other scientific equipment); or any other suitable substrates. By way of non-limiting illustration, some embodiments of the substrate include one or more medical implants or parts thereof.

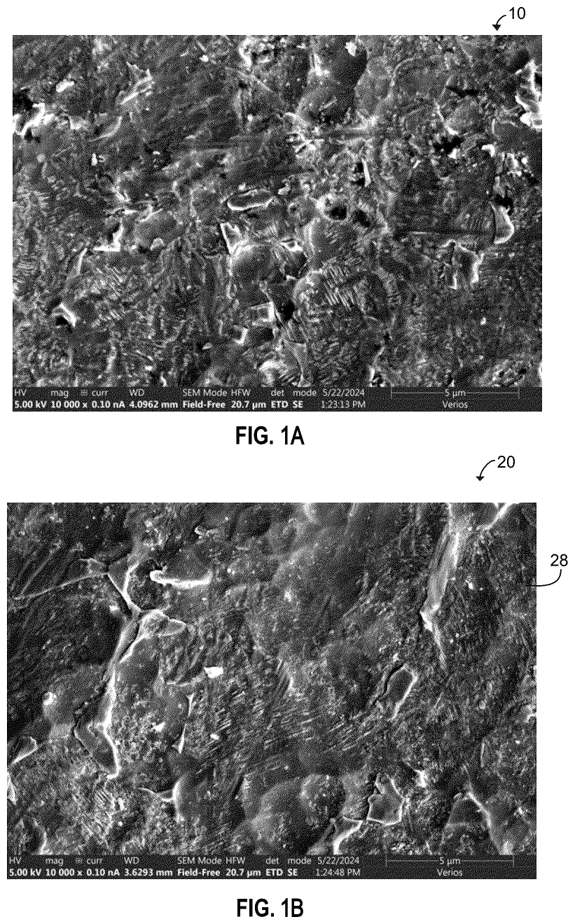

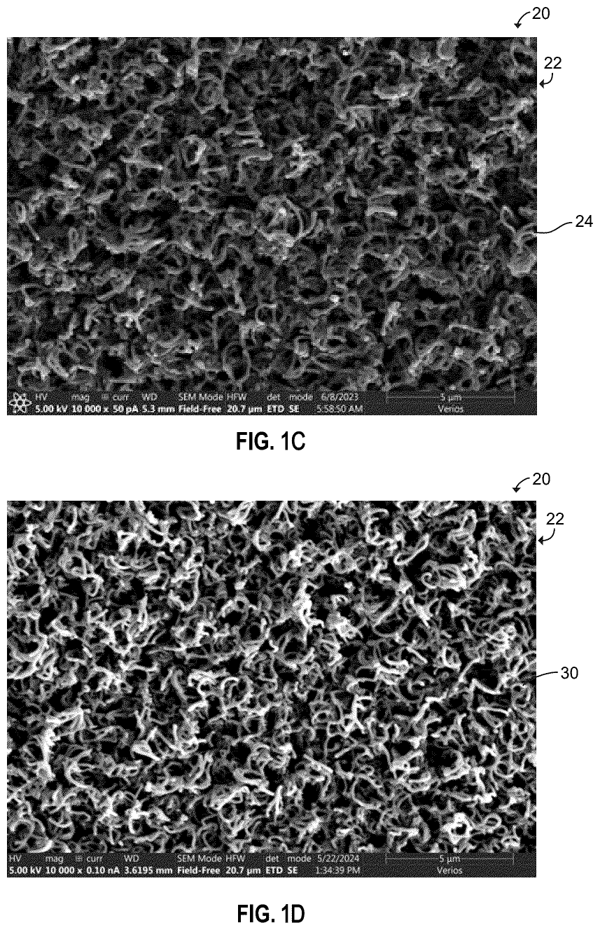

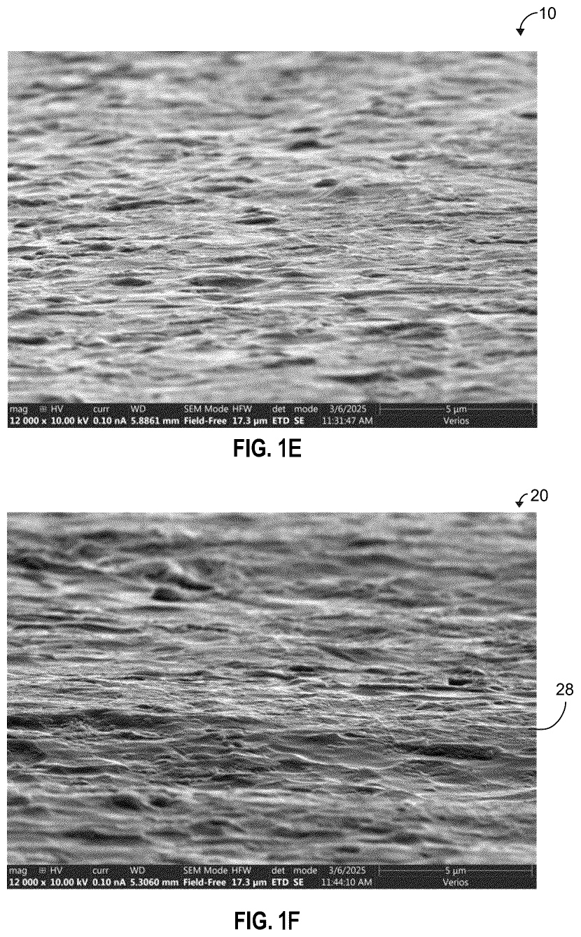

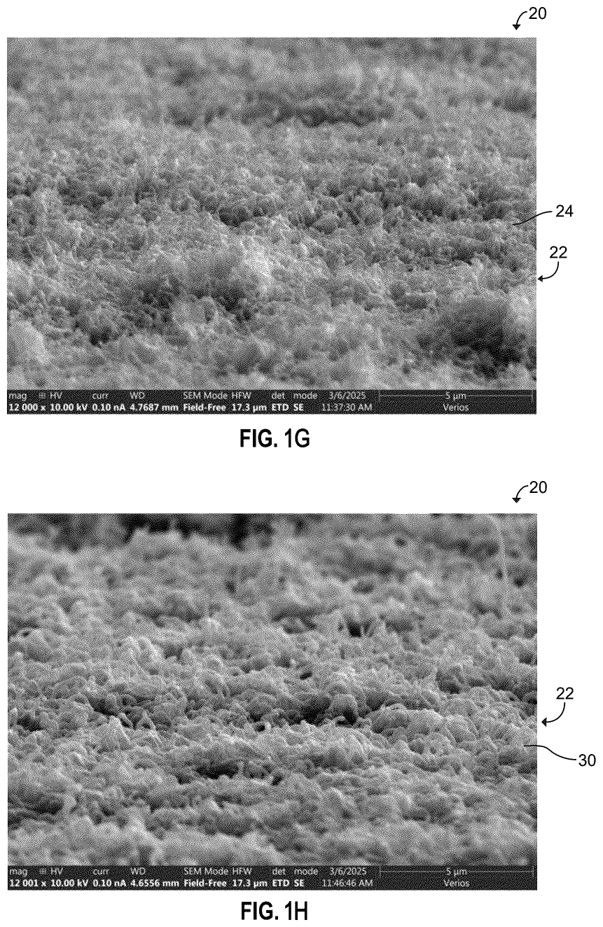

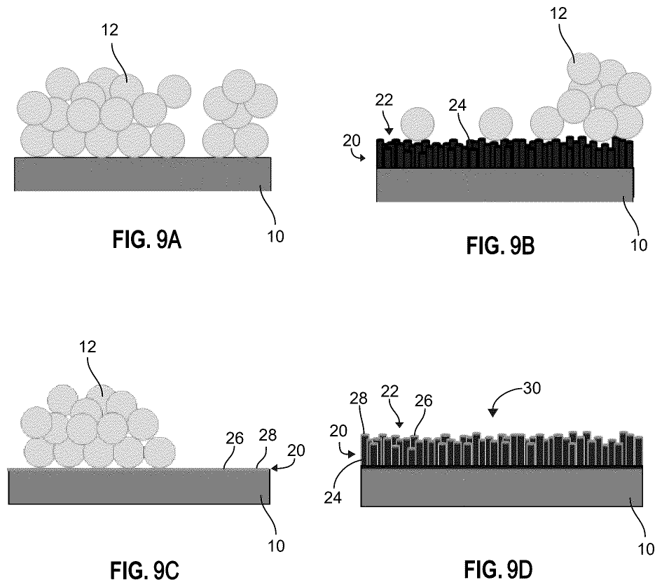

Although the substrate 10 can include any suitable material (e.g., wood, metal, metal alloy, glass, plastic, carbon fiber, polymer material, cardboard, paper, nylon, fabric, polyether ether ketone, polyethylene, polymethyl methacrylate, polytetrafluoroethylene, silicone, polyurethane flexible materials, rigid materials, partially rigid materials, or any other material or combination of materials), some embodiments are used in connection with a rigid or substantially rigid substrate, such as metal, glass, hard plastic, or another material generally configured to retain its shape. For example, many medical devices implement a metal material (e.g., titanium, titanium alloys, stainless steel, cobalt chromium alloys, nickel titanium alloys, tantalum, magnesium alloys, etc.), which may be particularly well suited to antibiofilm or antimicrobial coatings in accordance with some embodiments. Accordingly, some embodiments of the substrate include one or more surfaces comprising steel, titanium, Ti6Al4V alloy, one or more titanium alloys, or any other suitable metal substrate (or another rigid or substantially rigid substrate as may be used in connection with a medical device). By way of non-limiting illustration, FIGS. 1A, 1E, and 9A-D show SEM images of a titanium substrate 10 suitable for coating in accordance with some embodiments of the instant systems and methods.

According to some embodiments, the substrate 10 is coated with one or more coatings 20 configured to resist (e.g., slow, decrease the likelihood of, prevent entirely, or otherwise resist) the formation of biofilms 12 (e.g., one or more types of biofilms, most types of biofilms, substantially all types of biofilms, or even possibly all types of biofilms). According to some embodiments, the coating is antibacterial (killing, preventing the growth of, repelling, or otherwise counteracting one or more types of bacteria, such as Streptococcus, Enterobacteriaceae, and Pseudomonas species, as well as other types of bacteria, or other microorganisms. Thus, in some cases, the coating can help prevent osteomyelitis, bacteremia, or other infections. Indeed, in some cases, the coating is particularly useful against at least S. aureus and Pseudomonas aeruginosa (P. aeruginosa), both of which have a tendency to form biofilms that can be extremely difficult to remove or treat. By way of non-limiting illustration, FIGS. 1B-D, 1F-H, and 9B-D show some examples of suitable coatings 20.

In some implementations, the coating 20 (e.g., copper or any other suitable coating 20, as described below in more detail) is optionally applied directly to the substrate 10 (e.g., titanium or any other suitable substrate 10). Indeed, in some embodiments, the coating 20 is added directly to a metal substrate (such as a substrate comprising titanium or a titanium alloy). By way of non-limiting illustration, FIGS. 1B, 1F, and 9C show embodiments in which a copper coating 20 is disposed on a titanium substrate 10 (including, in some embodiments, without one or more carbon-infiltrated carbon nanotubes (CICNTs), which are discussed below).

According to some embodiments, the coating 20 includes one or more nanotextures 22 configured to prevent biofilm growth. The nanotexture can do so in any suitable manner, such as by killing the bacteria, preventing initial attachment of the bacteria, inhibiting growth of the bacteria, stabbing bacteria, decreasing the bacteria's ability to reproduce (e.g., by inhibiting cellular division), or otherwise. By way of non-limiting illustration, FIGS. 1C-D, 1G-H, 9B, and 9D show some examples of coatings 20 that include one or more antimicrobial nanotextures 22.

The nanotexture 22 can be formed of any suitable material. For example, the nanotexture can include one or more carbon-based structures (e.g., carbon nanotubes, carbon nanofibers, graphene sheets or structures, carbon nanowalls (single or multi walled), fullerenes, amorphous carbon structures, nanocrystalline carbon, carbon filaments, carbon quantum dots, nanodiamonds, or any other suitable carbon-based nanostructures); metal-based nanostructures (e.g., nanostructured titanium, stainless steel, gold, silver, copper, or any other suitable metals or alloys forming nanostructures); ceramic nanostructures (e.g., alumina nanotubes, zirconia nanowhiskers, titania nanorods, silicon dioxide nanofilaments, or any other suitable ceramic-based nanoelements); polymer-based nanoelements (e.g., electrospun nanofibers, dendritic polymers, functionalized polymer nanobristles, or any other suitable polymer nanostructures); biologically derived nanoarchitectures (e.g., cellulose nanofibers, chitosan nanospikes, collagen-based nanoassemblies, or any other suitable biologically derived or biomimetic nanostructures); any other suitable nanotexture, or any combination or hybrid of the foregoing. According to some embodiments, the nanotexture includes one or more CICNTs 24. By way of non-limiting illustration, FIGS. 1C, 1G, and 9B show nanotextures 22 that include CICNTs 24.

The nanotexture 22, such as the CICNTs 24 or other nanotexture components, can have any suitable topography configured to achieve the desired antimicrobial, antibiofilm, or other surface-functional properties. For example, the nanotexture can include one or more nanostructures having vertically aligned, interwoven, randomly oriented, entangled, branched, helical, coiled, layered, staggered, fractal, braided, or otherwise organized configuration(s) or combination of configurations. The CICNTs may be straight or curved, smooth-walled or rough-surfaced, hollow or partially filled, or otherwise configured, and they may include one or more junctions, bifurcations, kinks, nodes, bends, or other topographic features designed to enhance surface area, mechanical interlock, bacterial exclusion, or other desired interactions. In some embodiments, the nanotexture further includes one or more voids, channels, protrusions, pits, ridges, undulations, or any other suitable topographic feature defined by the disposition of the nanotubes, such that the resulting topography modifies bacterial adhesion, fluid dynamics, or mechanical compliance in a manner conducive to antimicrobial performance.

The nanotexture 22 can also have any suitable size. For example, some embodiments include some CICNTs 24 having a diameter (or width) between about 50 nm and 500 nm or any subrange thereof (e.g., 75 nm to 300 nm, 200 nm±150 nm, 150 nm±50 nm, 150 nm±25 nm, 150 nm±10 nm, or any other suitable diameter). The CICNTs can have any suitable wall thickness, such as between about 1 nm and 50 nm (e.g., 4 nm±3 nm, 10 nm±5 nm, 20 nm±10 nm, or any other suitable wall thickness). The CICNTs can also have any suitable length, such as between about 500 nm and 200 μm (e.g., 1 μm to 25 μm, 5 μm±3 μm, or any other suitable length).

According to some embodiments, the nanotexture 22 is configured to result in a substantial reduction in viable bacteria. For example, some embodiments of the nanotexture are configured to result in a reduction (compared to a control surface lacking the nanotexture) of at least 20%, 30%, 40%, 50%, 60%, 70%, or more of bacterial or other microorganisms.

According to some embodiments, the coating 20 includes one or more antimicrobial layers 26. Although in many cases the nanotexture 22 itself is antimicrobial (due to its particular structure), the antimicrobial layer 26 refers to one or more additional antimicrobial materials applied to the nanotexture (e.g., as a coating, film, disposition, or other covering) in a manner that (in some embodiments) generally preserves the structure of the nanotexture (although some embodiments may not preserve one or more aspects of the structure of the nanotexture).

The antimicrobial layer 26 can include one or more materials with various antimicrobial mechanisms of action, such as by causing membrane damage, producing reactive oxygen species, causing protein misfolding and aggregation, inducing cytotoxicity, or otherwise killing, inhibiting the reproduction of, or interfering with one or more microorganisms. For example, some embodiments of the antimicrobial layer include one or more of: copper 28 (e.g., copper ions, copper oxide, copper nanoparticles, copper alloys, or other copper-based agents); silver (e.g., colloidal silver, silver nitrate, silver sulfadiazine, silver nanoparticles, or other silver-based compounds); zinc-based compounds (e.g., zinc oxide, zinc chloride, zinc nanoparticles, or any other suitable zinc agents); gold (e.g., colloidal gold, gold nanoparticles, or any other suitable gold agents); selenium compounds; titanium dioxide; iron oxide; gallium compounds; magnesium-based antimicrobials; nitric oxide-releasing agents; ammonium compounds; biguanides (e.g., chlorhexidine, polyhexamethylene biguanide, or any other suitable biguanides); iodine-containing compounds; hydrogen peroxide or other peroxides; alcohols (e.g., ethanol, isopropanol, or any other suitable alcohol-based antimicrobials); antibiotics (e.g., vancomycin, gentamicin, ampicillin, cefepime, linezolid, ciprofloxacin, rifampin, clindamycin, metronidazole, erythromycin, tetracycline, daptomycin, mupirocin, or any other suitable antibiotic agents); natural antimicrobial compounds (e.g., chitosan, essential oils, eugenol, thymol, tannins, or any other suitable biologically derived agents); antimicrobial polymers (e.g., polyguanidines, polyethyleneimine, polyhexanide, or any other suitable polymeric agents); or any other suitable antimicrobial materials.

In some embodiments, the antimicrobial layer 26 includes copper 28. In such embodiments, the copper can be present in any suitable form, including as one or more elemental copper layers, copper alloys, copper oxides, copper salts, copper ions, copper nanoparticles, or any other suitable copper-containing compositions. That said, in some embodiments, the copper is deposited as a thin conformal film (e.g., using thermal evaporation, sputtering, electroplating, chemical vapor deposition, atomic layer deposition, physical vapor deposition, or any other suitable thin-film deposition techniques) in a manner that maintains or substantially preserves the underlying nanotexture 22 (e.g., the protrusions, gaps, porosity, and geometric features of CICNTs 24, or other nanostructures). The copper can be deposited in a continuous, semi-continuous, discontinuous, or any other suitable manner, and in some cases is applied as a layer having a thickness between about 1 nm and 50 nm (e.g., 1 nm to 20 nm, 8 nm±5 nm, 5 nm±3 nm, 5 nm±1 nm, or any other suitable thickness). Indeed, in some embodiments, the coper is applied as a layer having a thickness of 5 nm±2 nm.

Although the copper 28 can include polished copper, rolled copper, or copper otherwise deposited in any suitable manner, some embodiments include a copper thin film deposited using physical or chemical vapor deposition (which, in some cases, has a surface chemistry that has beneficial antimicrobial effects, particularly in connection with a nanotexture 22 as described herein). By way of non-limiting illustration, FIGS. 1B, 1F, and 9C show some embodiments of a copper 28 thin film coating 20.

In some cases, the antimicrobial layer 26 has a thickness that is smaller than the topological features (protrusions or processes, indentations or recesses, and other topological features) of the nanotexture 22. Indeed, in some cases, a thickness of approximately 5 nm—much thinner than may be used for some antimicrobial applications of copper 28—is surprisingly effective at augmenting antimicrobial copies of the CICNTs 24 or other nanotexture 22 to achieve particularly effective antimicrobial and antibiofilm results.

According to some embodiments, a nanotexture 22 that includes a CICNT 24 combined with an antimicrobial layer 26 that includes copper 28 has improved antimicrobial properties beyond the mere sum of the antimicrobial properties of CICNT and the antimicrobial properties of copper. Thus, a coating 20 that includes one or more copper-coated CICNTs (Cu-CICNTs) 30 can be surprisingly and remarkably effective at preventing or treating biofilms and infections that might otherwise afflict a non-coated substrate 10. By way of non-limiting illustration, FIGS. 1D, 1H, and 9D show some embodiments of Cu-CICNT 30 coatings 20.

In some embodiments, utilizing a Cu-CICNT 30 coating 20 (or other coating), the coating 20 is configured to reduce recoverable bacteria by at 50%. Indeed, in some embodiments, a Cu-CICNT 30 coating 20 (or other coating) is configured to reduce recoverable bacteria by at least 90% (e.g., at least 90%, 92%, 95%, 97%, 98%, 99%, 99.9%, 99.99%, 99.999%, 99.9999%, or any other suitable reduction) compared to an uncoated substrate 10 within any suitable amount of time (e.g., within 48 hours, 24 hours, 12 hours, 6 hours, or on any other suitable time frame). Indeed, in some embodiments, no viable bacteria are detected at or beyond a certain time threshold (e.g., 12 hours, 24 hours, 36 hours, or any other suitable time interval), either on the Cu-CICNT surface or in surrounding media (according to colony-forming unit (CFU) analysis or any other suitable viability assays).

According to some embodiments, the aforementioned reduction occurs across a wide variety of bacterial strains, such as S. aureus (in some cases, both methicillin-sensitive and methicillin-resistant isolates), P. aeruginosa, and other gram-positive and gram-negative biofilm-forming organisms. According to some embodiments, the Cu-CICNT 30 coating 20 achieves the intended effect through a combination of contact-dependent bacterial killing and surface-induced membrane disruption (in some cases, independent of any localized release of antimicrobial copper ions). In some cases, the antibacterial performance is maintained across a range of environmental conditions (e.g., in a variety of media, such as in saline, nutrient-rich broth, tissue-mimicking fluids, physiological conditions, or other environments), and in some cases it is sustainable over a defined time period (e.g., at least 24 hours, at least 48 hours, at least 1 week, at least 2 weeks, at least 1 month, at least 6 months, at least 1 year, or any other suitable amount of time) without measurable loss of activity or with minimal loss of activity (e.g., less than 1%, 2%, 5%, or any other suitable amount of loss of activity). In some cases, the Cu-CICNT 30 coating 20 (or other coating in accordance with this disclosure) is configured to retain its effectiveness under mechanical, thermal, fluidic, or any other type of stress, or in the presence of biological fouling or surface aging.

In some cases, the coating 20 is configured to operate based on its topological configuration. Accordingly, some embodiments are configured not to “run out.” In other words, some embodiments are configured to remain active (e.g., at least 99%, 95%, 90%, 85%, 80%, 75%, 65%, or 50% effective at reducing the viability of microorganisms) for an extended duration of time, such as for at least 1 month, 2 months, 3 months, 6 months, 9 months, 1 year, 2 years, 3 years, 5 years, 7 years, 10 years, 15 years, 20 years, 30 years, 50 years, or longer.

According to some embodiments, one or more methods for providing one or more surfaces with antibiofilm or antimicrobial characteristics are provided. The methods can be used in connection with any suitable substrate 10 (e.g., a medical implant, or any other substrate as discussed above), and can include one or more processes for forming one or more nanotextures 22, applying one or more antimicrobial layers 26, or otherwise preparing a functionalized surfaces to resist biofilm formation or microbial colonization. Moreover, one or more of the various portions of the described methods can be repeated, omitted, substituted, reordered, performed in parallel, performed in series, or otherwise modified in any suitable manner.

According to some embodiments, the described methods include obtaining a substrate 10. This can include purchasing, forming, manufacturing, or otherwise obtaining the substrate in any suitable manner.

In some cases, the methods include preparing the substrate 10, such as by cutting, shaping, polishing, or otherwise forming it into a desired geometry (e.g., an implant geometry, or any other suitable formation). In some cases, preparing the substrate includes cleaning the substrate (using any suitable cleaning technique, such as cleaning the substrate using isopropyl alcohol, ethanol, deionized water, detergent, acetone, ultrasonic bath, autoclave, UV, ethylene oxide, or any other cleaning agent or process).

According to some embodiments, preparing the substrate 10 includes surface-treating the substrate (e.g., with plasma, acid etching, corona discharge, or any other suitable technique) to enhance coating adhesion or otherwise prepare the substrate for coating 20. Indeed, some embodiments include sonicating the substrate in isopropyl alcohol or any other suitable cleaning agent (e.g., for 15 minutes or any other suitable duration), rinsing the substrate (e.g., in deionized water, or using any other suitable rinsing agent), and drying the substrate (e.g., through air-drying, heated drying, vacuum drying, or any other suitable drying technique).

In some embodiments, the methods include forming one or more nanotextures 22 on the substrate 10. Forming the nanotexture can be done in any suitable manner, such as via one or more of: nanotube growth (e.g., by chemical vapor deposition, plasma-enhanced chemical vapor deposition, thermal decomposition of a precursor gas, or otherwise); electrospinning (e.g., of polymer nanofibers, bioresorbable materials, hybrid blends, or any other similar process); nanolithography (e.g., electron beam lithography, nanoimprint lithography, soft lithography, or any other suitable patterning techniques); etching processes (e.g., reactive ion etching, plasma etching, wet chemical etching, or any other suitable form of etching); deposition techniques (e.g., electron beam deposition, oblique angle deposition, sputter deposition, layer-by-layer assembly of nanoparticles, electrophoretic deposition, sintering, or any other suitable form of deposition); laser ablation or any other form of laser structuring; templating against nanoporous molds (e.g., alumina templates, polymer stamps, or other molds); or any other suitable fabrication technique for forming a suitable nanostructure. Any suitable nanostructure (as described herein) can be thus formed.

In some embodiments, forming the nanotexture 22 includes forming one or more (and in some embodiments, many, or a large plurality of) CICNTs. Indeed, some embodiments include forming CICNTs via one or more nanotube growth processes. Although this can be done in any suitable manner, some embodiments include depositing a barrier layer onto the substrate (e.g., using electron-beam deposition or any other suitable deposition method). In accordance with some embodiments, the barrier layer includes Al₂O₃. Additionally, the barrier layer can be any suitable thickness, but in some embodiments it is between at least 10 nm and 1 mm thick, or any subrange thereof (e.g., between 20 nm and 2,000 nm, between at least 100 nm and 300 nm, 200 nm±50 nm, 200 nm±25 nm, or any other suitable subrange between 20 nm and 2,000 nm).

In some embodiments, the methods further include depositing one or more catalyst layers on top of (or otherwise in association with) one or more barrier layers (using thermal evaporation or any other suitable deposition method). While the catalyst layer can include any suitable composition, in some cases it includes iron. Additionally, the catalyst layer can be any suitable thickness, but in some embodiments it is between at least 0.1 nm and 10 μm thick, or any subrange thereof (e.g., at least 1 nm±0.1 nm thick, between 1 nm and 10 nm thick, 6 nm±1 nm thick, at least 6 nm±2 nm thick, or any other suitable subrange).

According to some embodiments, the methods include subjecting the substrate 10 (e.g., with the barrier layer and catalyst layer deposited thereon) to a carbon-infiltration process, such as by placing the substrate into a tube furnace under a controlled atmosphere (e.g., with flowing ethylene, acetylene, methane, or another carbon source gas). In some embodiments, the furnace is heated through a defined thermal cycle (e.g., 750° C., or any other suitable temperature, for 1 minute (or any other suitable amount of time) followed by 900° C. (or any other suitable temperature) for 7.5 minutes (or any other suitable time), or other suitable time/temperature profiles) to promote growth of CICNTs 24 with a desired morphology and distribution. For example, in some cases, CICNTs are prepared to have diameters of approximately 150 nm (e.g., ±1%, 2%, 3%, 5%, 10%, or 25%), or any other desired morphology and characteristics.

In some embodiments, the methods include depositing one or more antimicrobial layers 26 on the substrate 10, such as on a surface having the nanotexture 22. Such deposition can be done by any suitable technique (e.g., any of the techniques listed above). For example, some embodiments of the described methods include depositing a copper layer 28 (or any other suitable antimicrobial layer) using thermal evaporation (or any other suitable technique), such as using a filament-based physical vapor deposition system (e.g., an electric current runs through a crucible containing an evaporation source in a vacuum changer, with the current heating the evaporation source until it vaporizes, with the vaporized atoms then condensing onto a target material, forming a thin film coating). In some embodiments, the deposition is performed under vacuum using a voltage source (e.g., between 90 V and 150 V, or any subrange thereof, such as 120 V±10 V, or any other suitable voltage) to generate a suitable deposition rate. In some cases, the deposition rate is between 1 Å per second and 15 Å per second, or any subrange thereof (e.g., 5 Å±1 Å per second, or any other suitable subrange), until a target thickness (e.g., 5 nm±1 nm, or any other suitable thickness as discussed herein) is achieved.

According to some embodiments, the antimicrobial layer 26 is deposited conformally to preserve the geometry of the nanotexture 22.

The described systems and methods can be modified in any suitable manner. For instance, some embodiments include one or more additional layers. Such additional layers can include one or more additional antimicrobial components (e.g., antibiotics) or any other suitable components. Some embodiments have different coatings 20 for different surfaces (e.g., a Cu-CICNT 30 coating can be used on one surface of a medical implant that is particularly susceptible to biofouling, while a different surface that may experience more wear-and-tear may have a different coating to prevent breaking of CICNTs). Indeed, some embodiments include an implant with coating variations (e.g., less coating, no coating, different coating, or another variation) for high-wear areas, articulating surfaces, flexural zones, load-bearing zones, or other specific implant components.

As another modification, some embodiments include one or more features for preventing the nanotexture 22 from breaking or being released into the body (e.g., when included on an implant). For example, some embodiments include one or more coverings, such as meshes, grids, polymers, or other coverings configured to contain any portions of the nanotexture that separate from the substrate 10. Some embodiments include one or more adhesives configured to reinforce the nanotexture 22 or resist it separating from the substrate 10 in the event that it breaks.

In addition to the aforementioned features, the described systems and methods can include any other suitable feature. Indeed, while a coating 20 that includes just a nanotexture 22 or just an antimicrobial layer 26 can, in some cases, be effective at decreasing bacterial adhesion or biofilm formation, some embodiments that use a combined coating (e.g., a Cu-CICNT 30 coating 20) have vastly improved results, greatly decreasing—or even eliminating entirely—associated biofilms or bacterial infections. Some embodiments provide striking benefits, such as reproducibility (and the ability to coat uniformly across multiple batches), scalability (e.g., manufacturing scalability and application to a wide variety of different substrates), and biocompatibility.

### EXAMPLES

### Example 1: CICNT Preparation

Surfaces coated in CICNTs were prepared. Medical grade Ti6Al4V alloy (Ti) was cut from sheet stock into 9 mm squares (samples). Samples were sonicated in isopropyl alcohol for 15 minutes, rinsed in deionized water, and dried. For the creation of CICNT samples, 200 nm of Al₂O₃ was deposited on the surface of each sample as a barrier layer using electron-beam deposition. This was followed by the deposition of a 6 nm thin film of iron as a catalyst layer using a thermal evaporator. The prepared samples were then placed into a tube furnace for CICNT growth, with the infiltration step resulting in nanotubes with a final diameter of approximately 150 nm, which was confirmed using SEM.

### Example 2: Copper-Coated Sample Preparation

Copper (5 nm) was deposited on both bare Ti and CICNT samples using a thermal evaporator. A voltage of 120 V was used to generate a deposition rate of about 5 Å per second until 5 nm of copper had been deposited on the surface of each sample. This addition of copper did not substantially affect the surface topography of either type of sample, as shown by FIG. 1.

### Example 3: Bacterial Culture

Cultures of S. aureus were grown in shaking culture at 200 rpm for 16-18 hours in TSB. Cultures of P. aeruginosa were grown in shaking culture at 200 rpm for 16-18 hours in Luria-Bertani broth (LB). All cultures were then diluted to an optical density at 600 nm of 0.02 in Roswell Park Memorial Institute medium (RPMI) 1640 media (106 CFU), supplemented with sodium bicarbonate and HEPES (4-(2-hydroxyethyl)-1-piperazineethanesulfonic acid), as well as 2% heat-inactivated fetal bovine serum (FBS). Since pH plays a role in the solubility of copper ions, the pH was adjusted to 7.3 before sterile filtering the media. A CuCl₂ solution was used to add the appropriate amount of copper to the prepared RPMI media for experiments with exogenous copper ions. Then, 25 μL of inoculated RPMI was pipetted as a droplet onto the top of each sample. The droplets of bacterial culture were then incubated on the surface of each sample at 37° C. for the given amount of time. Each experiment was repeated at least three times, with each independent experiment being performed on at least two separate samples of each type.

### Example 4: Quantification of Bacteria in Biofilms, Bacterial Viability Assessment, Flow Cytometry

CFU analysis was performed. After incubation (see Example 3), the samples were washed once in sterile 1× phosphate-buffered saline (PBS) to remove unadhered cells. Samples were then removed to a clean well. Then, 500 μL of PBS was added to the sample surface and pipetted vigorously to dislodge the adherent cells in the biofilm. Once the cells from the biofilm were suspended in the PBS, 10 μL from each well were then removed and serially diluted in PBS before being spread on LB agar plates. The plates were then incubated at 37° C. for 24 hours, colonies were counted, and CFU counts were calculated.

To prepare the conditioned media, 500 μL of RPMI media rested on top of a copper-coated CICNT sample for 16 hours at 37° C. in a CO₂ incubator. Normal media from the same batch was used for comparison purposes, but without incubation on copper-coated CICNT. When testing the conditioned media, biofilms were grown under the same conditions as above. CFU analyses were also conducted and quantified the same as above.

Bacterial viability was assessed using the BacLight™ RedoxSensor™ Green Vitality Kit, which includes two reagents: Propidium Iodide (PI) and RedoxSensor™ Green (RSG). PI (20 mM in DMSO) is excluded by bacterial cells with intact membranes but penetrates bacteria with compromised membranes, binding to DNA and thus serving as a marker of non-viable or dead bacterial cells. In contrast, RedoxSensor™ Green reagent stain (1 mM in DMSO, and diluted 1:10 in PBS prior to use) penetrates live bacterial cells and fluoresces green if the bacteria are alive. Using this viability assessment, biofilms were grown on samples for 6 hours and then disrupted by vigorous pipetting with 500 μL PBS. As a positive control for membrane-compromised cells, 2 μL of the electron transport chain uncoupler carbon cyanide m-chlorophenyl hydrazone (CCCP, 10 μM) was added to approximately 10⁶ healthy bacteria in 500 μL of PBS, followed by a 5-minute incubation at room temperature. Healthy untreated bacteria served as live, membrane-intact controls. Unstained controls were also used to facilitate compensation and gating. For staining, 1 μL RedoxSensor™ Green reagent and 1 μL of PI were added to each 500 μL bacterial suspension, gently vortexed, and incubated at room temperature in the dark for five minutes. Immediately before analysis, counting beads were added to each sample to normalize volume acquisition. Samples were analyzed using a CytoFLEX flow cytometer.

Gating was performed using the positive, negative, and unstained controls according to the manufacturer's guidelines. Bacterial populations were initially identified using forward scatter (FSC) and side scatter (SSC) on logarithmic scales. FSC was used to assess cell size, while SSC indicated internal complexity. Debris was identified as events with markedly lower FSC and SSC values, falling outside the main bacterial population gate. Data were processed and analyzed using FlowJo software (version 10.6.2).

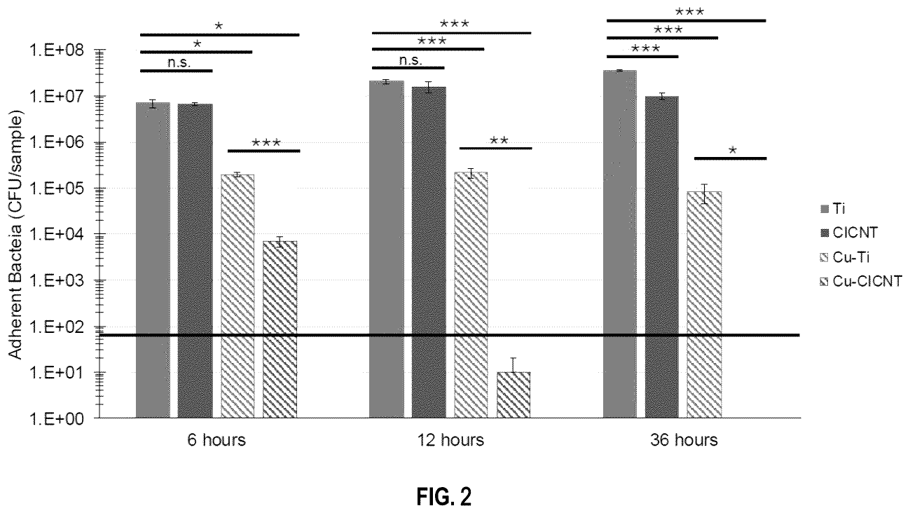

Example results can be seen in FIGS. 2-8, as well as in the following examples. Generally speaking, CFU analysis demonstrated that there was a synergistic effect between the surface topography and the presence of copper, resulting in up to a 6.9-log (99.9999%) reduction in the number of bacteria present by 12 hours.

### Example 5: Effect of Cu-CICNT

To determine the effect of a copper thin film combined with the CICNT surface, RPMI inoculated with the JE2 strain of S. aureus (MRSA) was incubated for 6, 12, or 36 hours on bare Ti, CICNT, copper-coated Ti (Cu—Ti), and copper-coated CICNT (see FIG. 2). At 6 hours, there was no significant difference in the number of adherent bacteria retrieved from Ti or CICNT, but there was a 1.5-log (97%) reduction in the number of bacteria recovered from Cu—Ti (p=0.0022) and a 3-log (99.9%) reduction in the number of bacteria recovered from the Cu-CICNT surface (p=0.002) (see FIG. 2). By 12 hours, there was still no significant difference in the number of adherent bacteria retrieved from Ti and CICNT, but there was a 2-log (99%) reduction in the number of bacteria recovered from Cu—Ti (p=1.4×10−6) and a 6.3-log (99.9999%) reduction in the number of bacteria recovered from Cu-CICNT (p=6.1×10−6) (see FIG. 2). The average number of adherent bacteria recovered from the Cu-CICNT surface by 12 hours was below the limit of detection for the plate counting assay. At 36 hours there was a significant decrease in the number of bacteria on Ti and CICNT (72% reduction, p=<0.0001), and a significant decrease in the number of bacteria on Cu—Ti (2.6-log (99.8%) reduction, p<0.0001). At 36 hours, no bacteria were recovered from any of the Cu-CICNT samples, a 7.5-log reduction (p<0.0001).

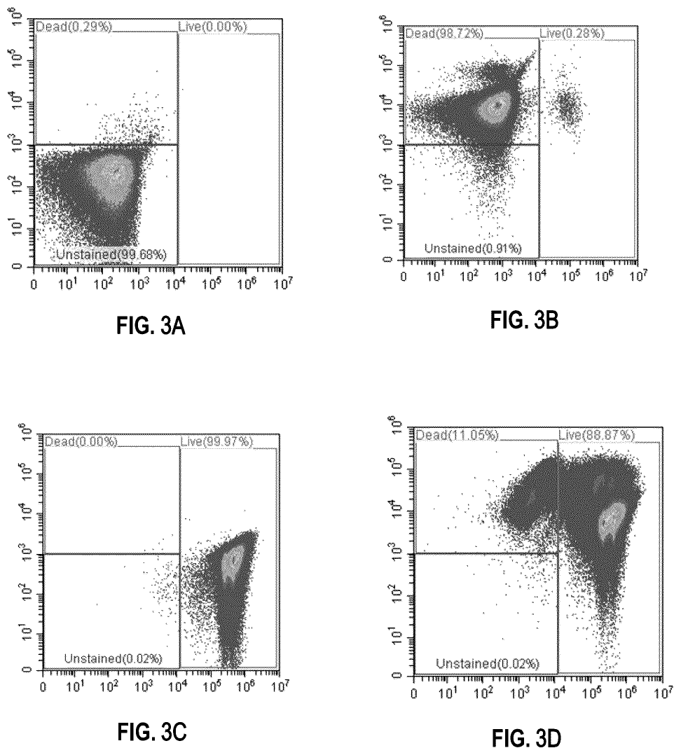

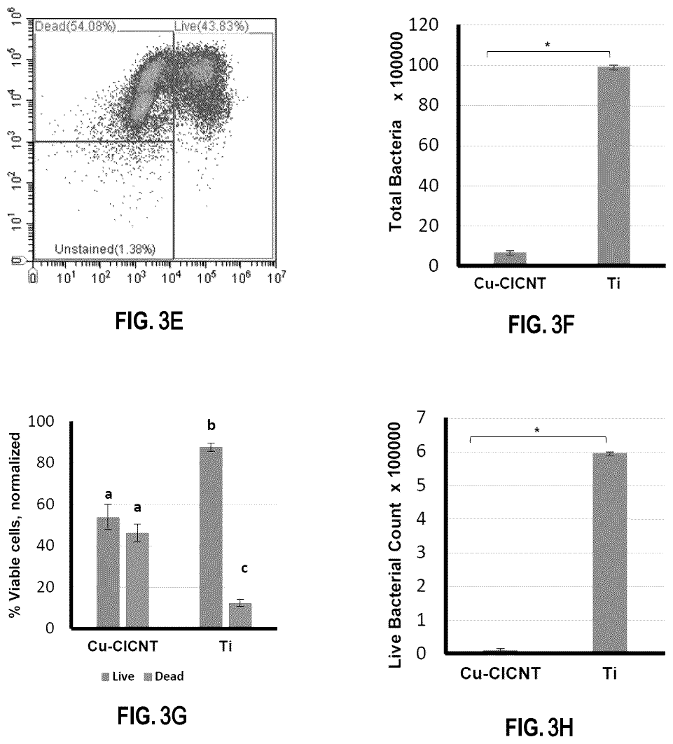

S. aureus biofilms were grown on either Ti or Cu-CICNT surfaces for 6 hours. At this time point, live bacteria were present on both surfaces (a ˜3-log reduction on Cu-CICNT by CFU analysis), allowing us to detect both live and dead stained cells. The biofilms were removed, along with any unattached cells from each sample, and stained with RedoxSensor™ Green and propidium iodide. Flow cytometry was performed using bead-based normalization to standardize event counts (see FIG. 3). Significantly fewer bacterial events were detected in the same volume of samples collected from Cu-CICNT surfaces compared to Ti (14-fold difference, p=0.0001). To assess the relative proportions of live and dead cells, total events were normalized such that the sum of live and dead cell percentages equaled 100% (see FIG. 3G). On the Ti surface, about 12.4+/−1.71% of recorded, bead-normalized cells stained positive for propidium iodide, indicating that the cells no longer had an intact membrane, and they were categorized as dead (see FIGS. 3D and 3G). These results are comparable to cell viability assessed on CICNT without a copper coating. In contrast, Cu-CICNT samples exhibited a significantly higher proportion of PI+/dead cells (46.2+/−4.10%, a 4-fold increase compared to Ti, p=0.0007; see FIGS. 3E and 3G). These results suggest that bona fide cell death occurs upon exposure to Cu-CICNT, rather than cells existing in a viable but unculturable state, which has been reported previously when bacteria are exposed to copper. Additionally, significantly higher absolute numbers of living cells were recovered from Ti surfaces than Cu-CICNT surfaces (a ˜50-fold difference, p=0.0008) (see FIG. 3H).

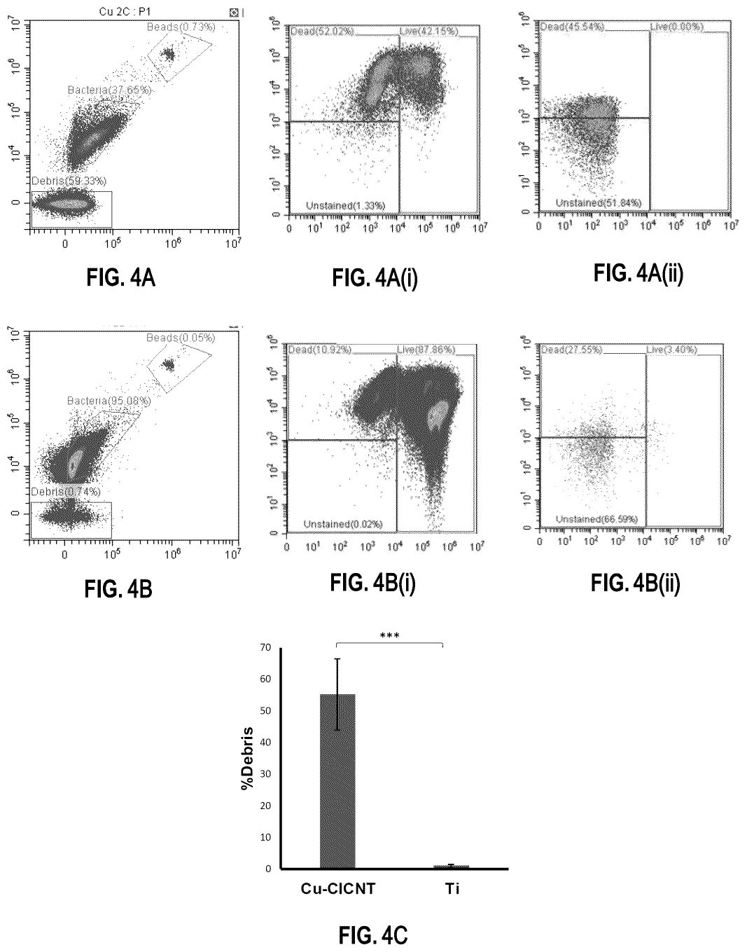

Using an analysis of FSC and SSC populations, significantly more debris-sized events were recorded for the Cu-CICNT surfaces than for the Ti surfaces (55.1+/−11.2% of events vs 0.92+/−0.62%, p=0.002) (see FIG. 4). This may suggest that some of the bacteria on the Cu-CICNT surface have been lysed, which would result in smaller, debris-sized fragments detected by flow cytometry. These events may represent subcellular debris due to the smaller size and lower complexity. To further characterize the debris population, events were gated based on PI and RedoxSensor™ Green staining as described previously. The majority of debris was either unstained or PI-positive but negative for RedoxSensor™ Green, suggesting that these events likely represent dead or non-viable material (see FIGS. 4A(ii) and 4B(ii)).

### Example 6: Effect on Alternative Isolates

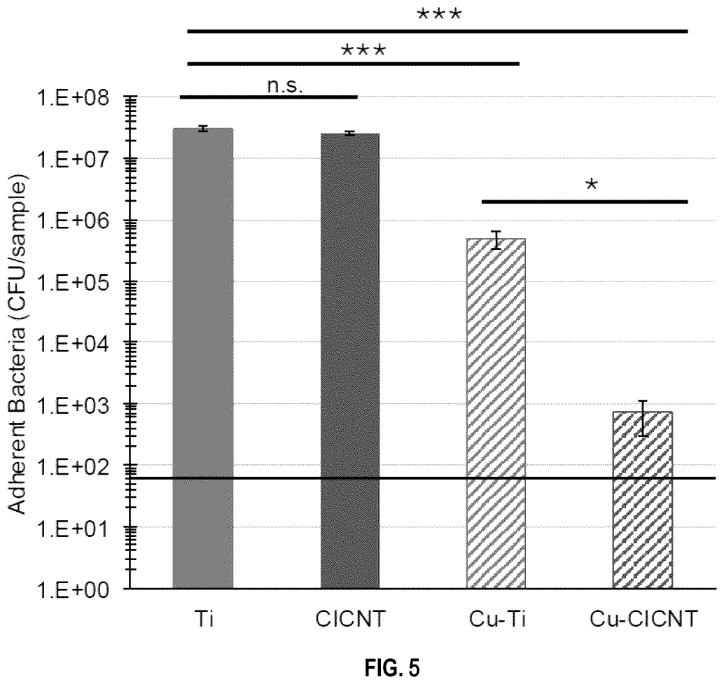

Biofilm reduction is observed with a different isolate of S. aureus. In this regard, different isolates of the same bacterial species may react differently to antibacterial treatments. For this reason, a second isolate of S. aureus was tested on the Cu-CICNT surface. Indeed, RPMI inoculated with the SH1000 strain of S. aureus (MSSA) was incubated for 12 hours on bare Ti, CICNT, Cu—Ti, and Cu-CICNT. There was no significant difference in the number of adherent bacteria on Ti vs CICNT (p=0.13), but there was a 1.7-log (98%) reduction in the number of bacteria on Cu—Ti compared to Ti (p=0.00032), and a 4.6-log (99.99%) reduction on Cu-CICNT (p=0.0092) (see FIG. 5).

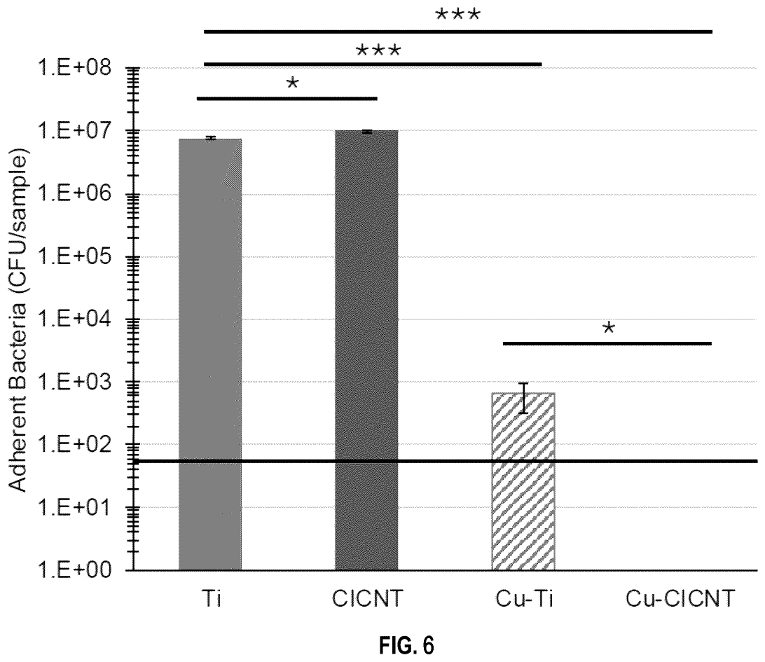

Similarly, biofilm reduction is observed with P. aeruginosa. In this regard, S. aureus is a gram-positive bacterium. Gram-positive and gram-negative bacteria may react differently to antimicrobial surfaces due to differences in their cell walls and membrane structures. Gram-positive bacteria can have a thick, relatively stiff layer of peptidoglycan surrounding the cells, while gram-negative bacteria can have a thinner layer of peptidoglycan sandwiched between two flexible membranes. P. aeruginosa was used to test the response of a gram-negative bacterium to the Cu-CICNT surface since this organism is also very adept at forming biofilms on implanted devices. RPMI inoculated with strain 15442 of P. aeruginosa was incubated for 12 hours on bare Ti, CICNT, Cu—Ti, and Cu-CICNT. There was a small but significant difference in the number of adherent bacteria on Ti and CICNT (20% increase, p=0.0087) (see FIG. 6). However, the differences in the number of adherent bacteria on Cu—Ti (4.2-log (99.99%) reduction, p=0.037) and Cu-CICNT (6.9-log (99.9999%) reduction, p<5×10−9) were much greater (see FIG. 6).

### Example 7: Effect Not Solely Due to Copper Ions

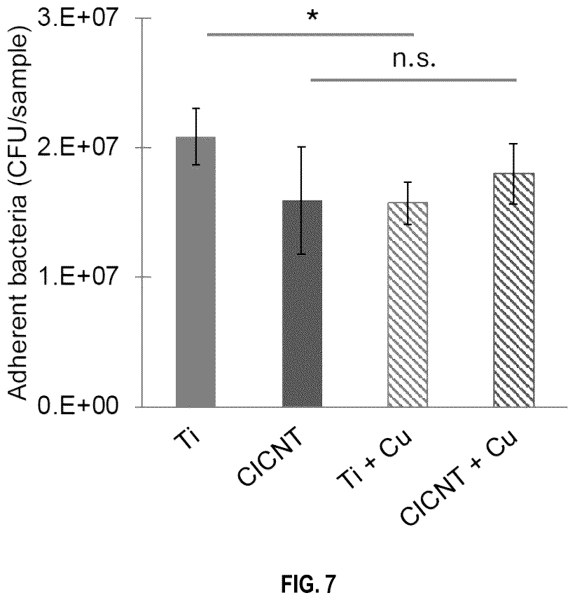

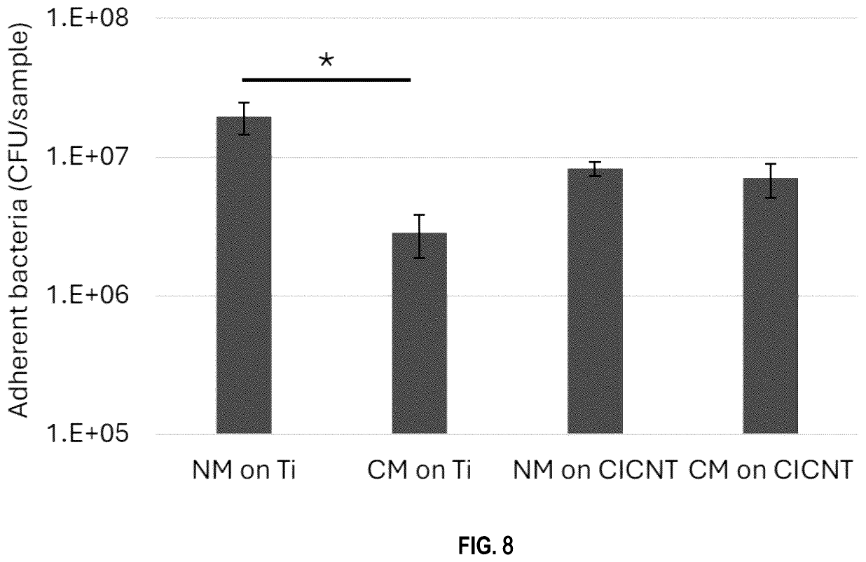

To determine whether biofilm reduction was simply due to the concentration of copper ions in the solution, 6.2 ppm of exogenous copper was added to the bacterial media using a CuCl₂ solution. S. aureus bacteria (JE2 strain) were grown in the media containing additional copper ions on non-copper-coated Ti or CICNT for 12 hours and then enumerated. There was a small but significant decrease in adherent bacteria on Ti+copper ions vs Ti alone (25% reduction, p=0.04), and no significant decrease in bacteria on CICNT vs CICNT+copper ions (p=0.8) (see FIG. 7). To better understand if the antibiofilm effect was mediated by something released into the growth media after exposure to the Cu-CICNT surface, conditioned media were produced by incubating RPMI media in the presence of Cu-CICNT for 16 hours. This conditioned media was used to culture S. aureus (JE2) bacteria on a non-copper-coated Ti surface for 12 hours, alongside a Ti surface with normal media. After quantification of CFU, a significant reduction was observed in bacterial counts on Ti with conditioned media vs Ti with normal media (85.4% reduction, p=0.0039) (see FIG. 8). This result suggests that copper ions released into solution exhibit a modest bactericidal effect, but it cannot explain the dramatic effects seen after direct exposure to a Cu-CICNT surface. Interestingly, there was no significant difference between conditioned media and normal media when cells were grown on a CICNT surface (14.9% reduction, p=0.28), and this result is similar to the results in FIG. 7 where CICNT also failed to show an effect mediated by CuCl₂ media.

Any and all of the components in the figures, embodiments, implementations, instances, cases, methods, portions, instantiations, examples, applications, iterations, and other parts of this disclosure can be combined in any suitable manner and via any suitable permutation. Additionally, any component can be removed, separated from other components, modified with or without modification of like components, or otherwise altered together or separately from anything else disclosed herein.

As used herein, the singular forms “a”, “an”, “the”, and other singular references include plural referents, and plural references include the singular, unless the context clearly dictates otherwise. For example, reference to a layer includes reference to one or more layers, and reference to CICNTs includes reference to one or more CICNTs. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. An antibiofilm, antimicrobial coating comprising:
a nanotexture having an antibacterial topology; and
an antimicrobial layer formed on the nanotexture, the antimicrobial layer having antibacterial characteristics.

2. The antibiofilm, antimicrobial coating of claim 1, wherein the nanotexture comprises a plurality of carbon-infiltrated carbon nanotubes.

3. The antibiofilm, antimicrobial coating of claim 1, wherein the nanotexture comprises a carbon-infiltrated carbon nanotube with a diameter of between 100 nm and 200 nm.

4. The antibiofilm, antimicrobial coating of claim 1, wherein the antimicrobial layer comprises a metal.

5. The antibiofilm, antimicrobial coating of claim 1, wherein the antimicrobial layer comprises copper.

6. The antibiofilm, antimicrobial coating of claim 1, wherein the antimicrobial layer comprises copper deposited via physical vapor deposition.

7. The antibiofilm, antimicrobial coating of claim 1, wherein the antimicrobial layer has a thickness of between 1 nm and 10 nm.

8. The antibiofilm, antimicrobial coating of claim 1, wherein the antimicrobial coating is formed on a substrate that comprises a metal.

9. The antibiofilm, antimicrobial coating of claim 1, wherein the antimicrobial coating is configured to reduce both Staphylococcus aureus and Pseudomonas aeruginosa viability.

10. The antibiofilm, antimicrobial coating of claim 1, wherein the antimicrobial coating is configured to remain active for at least 1 month.

11. A method for forming an antibiofilm, antimicrobial coating, the method comprising:
forming a nanotexture on a substrate, the nanotexture having an antibacterial topology; and
forming an antimicrobial layer on the nanotexture, the antimicrobial layer having antibacterial characteristics.

12. The method of claim 11, wherein the forming the nanotexture on the substrate comprises:
depositing a barrier layer on a surface of the substrate;
depositing a catalyst layer on the barrier layer; and
subjecting the substrate to a carbon-rich environment to form a plurality of carbon-infiltrated carbon nanotubes.

13. The method of claim 11, wherein the forming the antimicrobial layer on the nanotexture comprises deposing a conformal copper coating over the nanotexture.

14. The method of claim 11, wherein the forming the antimicrobial layer comprising deposing a conformal copper coating using thermal evaporation.

15. The method of claim 11, wherein the substrate comprises a medical implant.

16. A medical implant comprising:
a surface, the surface having an antibiofilm, antimicrobial coating comprising:
a nanotexture having an antibacterial topology; and
an antimicrobial layer formed on the nanotexture, the antimicrobial layer having antibacterial characteristics.

17. The medical implant of claim 16, wherein the surface comprises a metal.

18. The medical implant of claim 16, wherein the nanotexture comprises a carbon-infiltrated carbon nanotube.

19. The medical implant of claim 16, wherein the antimicrobial layer comprises copper.

20. The medical implant of claim 16, wherein the nanotexture comprises a carbon-infiltrated carbon nanotube and the antimicrobial layer comprises copper.
